(19) United States
(12) Patent Application Publication
(10) Pub. No.: US 2008/0178412 A1
(43) Pub. Date: Jul. 31, 2008

(54) CLEANING SYSTEM FOR SHOPPING CARTS

(76) Inventor: MICHAEL A. KITER, Yadkinville, NC (US)

Correspondence Address: VINCENT TASSINARI, 125 GRAND AVE., SUITE 201, LONG BEACH, CA, 90803, US

(21) Appl. No.: 11/669,541

(22) Filed: Jan. 31, 2007

(51) Int. Cl. B60S 3/04

(52) U.S. Cl. 15/309.2; 15/21.1; 15/3; 15/3.51; 15/319; 15/4

(57) ABSTRACT

This patent discloses a cleaning system for shopping carts. The cleaning system may include a prewash station, a wash station, a rinse station, a drying station, a physical disinfecting station, a conveyor, and a recovery tank. The wash station may include brushes. The drying station may include a fan configured to blow a thin sheet of air downward and onto the shopping cart. The physical disinfecting station may include an ultraviolet light generator. The conveyor may include a track system having hooks attached to chains that are configured to be moved by a conveyor motor. A frame may surround and support the stations. The cleaning system additionally may include a water recovery/supply system and an external spray wash hose attached to a reservoir to pre-prewash the shopping carts.

### BACKGROUND

1. Field of Endeavor

The information disclosed in this patent relates to cleaning shopping carts, where the shopping carts may be moved from one station to the next.

2. Background Information

A shopping cart is a cart supplied by a shop, especially a supermarket, for use by customers inside the shop to transport merchandise to the check-out counter, and, after paying, often also to their car in an adjacent, uncovered parking lot. Called a buggy or a trolley in British English, a carriage or shopping carriage in the United States (U.S.) region of New England, and a bascart in other regions of the U.S. customers typically are allowed to leave the carts in the car parking lot, and store personnel return the carts to the shop.

Bird droppings carry thousands of know and unknown diseases. For whatever reason, birds seem to target shopping carts left out in an uncovered parking lot. In addition, customers drop or spill food into shopping carts that eventually may grow germs such as salmonella. One way or another, these shopping carts become contaminated and carry a host of infectious agents.

Everyday, millions of shoppers handle these dirty shopping carts and expose themselves to germs, viruses, and infectious diseases. This is especially dangerous to pregnant women as there are at least five situations of unborn babies prematurely dying due to the pregnant woman's exposure to bird droppings on shopping carts.

The person utilizing the contaminated shopping carts is not the only one at risk. The general public also is a risk to inadvertent exposure due to the spreading of infectious diseases derived from the use of contaminated shopping carts. For many young or old, contaminated shopping carts may be a death sentence.

Bird or food droppings left on a shopping cart pose a risk of serious illnesses or even death. However, a number of techniques have been employed to clean shopping carts. Some stores provide sanitation wipes for customers to clean their shopping cart. Moreover, some customers have their own fabric covers that fit over the handles of the shopping cart. In addition, there are shopping cart child seat covers made of fabric to keep their children clean and germ free. However, in each instance, there still are portions of a contaminated shopping cart that pose risks to users. Some patents do disclose cleaning system for shopping carts, but these systems are inefficiency, difficult to operate, inadaptable, and may not reach a sufficient level of hygiene.

What is needed is a cleaning system for shopping carts to overcome these and other problems.

### SUMMARY

This patent discloses a cleaning system for shopping carts. The cleaning system may include a prewash station, a wash station, a rinse station, a drying station, a physical disinfecting station, a conveyor, and a recovery tank. The wash station may include brushes. The drying station may include a fan configured to blow a thin sheet of air downward and onto the shopping cart. The physical disinfecting station may include an ultraviolet light generator. The conveyor may include a track system having hooks attached to chains that are configured to be moved by a conveyor motor. A frame may surround and support the stations. The cleaning system additionally may include a water recovery/supply system and an external spray wash hose attached to a reservoir to pre-prewash the shopping carts.

### BRIEF DESCRIPTION OF THE FIGURES

FIG. 1 is a side elevated schematic view of a cleaning system 100 for shopping carts 10.

FIG. 2 is a method 300 to operate the cleaning system 100 for shopping carts 10.

### DETAILED DESCRIPTION

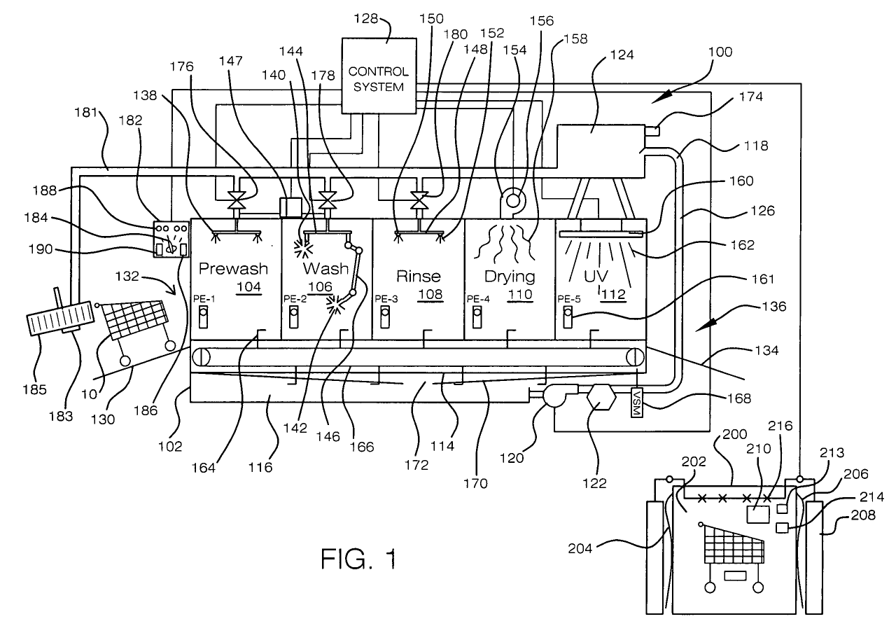

FIG. 1 is a side elevated schematic view of a cleaning system 100 for shopping carts 10. Cleaning system 100 may protect users of shopping carts 10 from exposure to germs, viruses, and infectious diseases. In addition, cleaning system 100 may render shopping carts 10 clean of bird droppings landing on a shopping cart 10 and food that has been drop or spilled into a shopping cart 10.

Cleaning system 100 may include a frame 102 housing a prewash station 104, a wash station 106, a rinse station 108, a drying station 110, a physical disinfecting station 112, a conveyor 114, a recovery tank 116, a water recovery/supply system 118 having a pump 120, a filter 122, a reservoir 124, and piping 126. Cleaning system 100 further may include a control system 128.

Frame 102 may be an elongated, rigid structure assembled to surround and support prewash station 104, wash station 106, rinse station 108, drying station 110, physical disinfecting station 112, conveyor 114, and recovery tank 116. Frame 102 may have a frame entrance ramp 130 at a first end 132 and a frame exit ramp 134 at a second end 136. Frame 102 may be made of stainless steel, aluminum, or the like.

Prewash station 104 may include a series of high pressure water jets 138. The high pressure water jets 138 may contact shopping carts 10 with a water/soap solution to loosen adherent dirt, impurities, foreign matter, and any other undesired matter from shopping carts 10.

Wash station 106 may feature a first brush 140 and a second brush 142 that may mechanically scrub all or a portion of a shopping cart 10 and squirt water as well. Each brush 140, 142 may be attached to a water station head 144 through one or more links 146. For example, brush 140 may be attached to water station head 144 through one link 146 and brush 142 may be attached to water station head 144 through four links 146. Each link 146 may move relative to the other links 146 connected between a brush and the water station head 144.

Control system 128 may independently control each link 146 to move first brush 140 and second brush 142 along predetermined paths. With the shape and size of shopping cart 10 standardized and known, control system 128 may be programmed to move first brush 140 and second brush 142 along the entire surface of shopping cart 10. Each brush 140, 142 may be rotated and water or other cleaning fluid may be dispensed from brushes 140, 142. Thus, wash station 106 may transfer scrubbing work and fluid to shopping carts 10 to remove adherent dirt, impurities, foreign matter, and any other undesired matter from shopping carts 10. A cleaning fluid tank 147 may be attached to both prewash station 104 and wash station 106. Cleaning fluid tank 147 may be partitioned and each compartment may include cleaning fluid such as liquid soap to be added to prewash station 104 and wash station 106.

Rinse station 108 may be a high pressure, hot water rinse system having a rinse station head 148. Rinse station head 148 may have a first spray nozzle 150 and second spray nozzle 152. Rinse station head 148 may be configured to rotate as rinsing water exits spray nozzles 150, 152. This may contact shopping carts 10 with liquids to wash away any soap or debris added to shopping carts 10 from wash station 106.

Drying station 110 may include an air knife drying system 154. Air knife drying system 154 may include a fan 156 and be configured to provide wide area coverage with an air curtain 158. Air curtain 158 may be a thin sheet of air blown downward. As a shopping cart 10 perpendicularly passes through air curtain 158, water may be forced downward off shopping cart 10 to dry shopping cart 10.

Physical disinfecting station 112 may include an ultraviolet light generator 160 configured to produce ultraviolet light 162 to disinfect shopping carts 10. Disinfection may be the result both of the intensity (I) of ultraviolet light 162 and the length of time (T) ultraviolet light 162 resides on each surface of shopping cart 10. This intensity (I) multiplied by the time (T) may represent the dose of ultraviolet light 162, usually expressed in mill Joules per centimeter squared (mJ/cm²). Table I below sets out the dose requirements for acceptable reduction in the number of common live microorganisms.

TABLE I

| Species | Dose (mJ/cm²) |
|---|---|
| Legionella Pneumophilla | 2.0 |
| Streptococcus feacalis | 4.5 |
| Clostridium tetani | 4.9 |
| Pseudonomas aeruginosa | 5.5 |
| Saccharomyces cervisiae | 6.0 |
| Hepatitis A virus | 11.0 |
| Bacillus subtilis (spore) | 12.0 |
| Hepatitis Poliovirus | 12.0 |
| Infectious pancreatic necrosis | 60.0 |

Experiments also have shown to reduction in number of live Escherichia coli (E. coli) microorganisms by 99.9% requires an ultraviolet light dose of 16.2 mJ/cm². While an ultraviolet light dose of 60.0 mJ/cm² may be sufficient to disinfect shopping cart 10 of most common live microorganisms, this would require an unsafe, high intensity (I) ultraviolet light 162 or a very slow moving conveyer 114. However, if the dose were too low, most common live microorganisms may survive physical disinfecting station 112. Thus, in one example, a dose of ultraviolet light 162 applied against each surface of shopping cart 10 may not be less than 16.2 mJ/cm² to kill most living microorganisms and may not be greater than 20.0 mJ/cm² to keep conveyor 114 moving at a reasonable speed.

Each station 104, 106, 108, 110, and 114 may include a light sensor 162, such as light sensors PE-1, PE-2, PE-3, PE-4, and PE-5. Each light sensor 162 may identify whether a shopping cart 10 is located within the station associated with the light sensor 162 and provide information to control system 128 to permit control system 128 to identify a horizontal position of shopping cart 10 relative to each station 104, 106, 108, 110, and 114.

As noted above, other parts of cleaning system 100 may include conveyor 114. Conveyor 114 may be located in a lower portion of cleaning system 100 and may feature a track system, the center of which may be equipped with a chain-based conveyor system that may pull shopping carts 10 through cleaning system 100. Shopping carts 10 may be manually or mechanically pushed or pull through frame 102 of cleaning system 100. Conveyor 114 may include hooks 164 attached to chains 166 that may be moved by a conveyor motor 168. Chains 166 may be endless and conveyor motor 168 may be a variable speed motor (VSM) having a horizontal axis (not shown).

Recovery tank 116 may be positioned in a lower portion of cleaning system 100 to receive debris, drippings, and other fluid from stations 104, 106, 108, 110, and 114. A drip pan 170 that may extend a length and a width of stations 104, 106, 108, 110, and 114 may angle down to a drain hole 172 to feed recovery tank 116. In an example, drip pan 170 may extend a length and a width of stations 104, 106, 108, and 110 only.

Water recovery/supply system 118 may supply clean, high pressure water to prewash station 104, wash station 106, and rinse station 108. As noted above, water recovery/supply system 118 may include pump 120, filter 122, reservoir 124, and piping 126

Pump 120 may be any mechanical device that may move fluid by pressure or suction. In addition, pump 120 may be configured to separate liquids from solids. Pump 120 may be a high pressure pump located at some point within or on frame 102. Preferably, pump 120 may be located to an outlet of recovery tank 116. The separated liquids and the separated solids maybe recovered in whole or in part for subsequent use.

Filter 122 may receive liquids from pump 120 and remove impurities from that portion of the liquid that is water. Filter 122 may be positioned between pump 120 and reservoir 122.

Reservoir 124 may be a tank configured to collect and store liquid for use by prewash station 104, wash station 106, and rinse station 108. Reservoir 124 may receive clean water from filter 122 and receive clean water through a reservoir valve 174 attached to reservoir 124. Each of prewash station 104, wash station 106, and rinse station 108 may include a station valve 176, 178, 180, respectively, to control the flow and pressure of water delivered from reservoir 124. Piping 126 may transport fluid between elements of water recovery/supply system 118. Reservoir 124 may be under pressure from pump 120 and this fluid pressure may cause first brush 140 and second brush 142 of wash station 106 to rotate.

A spray wash hose 181 may be attached to reservoir 124 to pre-prewash shopping carts 10. Spray wash hose 181 may be external to frame 102 and include a hose sprayer 183 and a scrub brush 185 surrounding hose sprayer 183. Spray wash hose 181 may be utilized on a heavily soiled shopping cart 10 to remove bird droppings, rotting meat, and other adhering debris.

Control system 128 may remotely manage the behavior of other device in cleaning system 100 through electronic signals transmitted as a result of signals from a microprocessor (not shown). Control system 128 may receive user input from a control switch box 182. Control switch box 182 may have a control knob 184 to dial in various mode of operations (or cleaning modes), a key pad 186 to enter access codes and number of carts, and displays such as status indicator lights 188 and number of carts display 190. Control switch box 182 may be mounted on frame 102 at a location adjacent to first end 132.

Table II below lists the cleaning modes that may be set through control switch box 182:

TABLE II

| Mode | Prewash 104 | Wash 106 | Rinse 108 | Drying 110 | UV 112 | Mist 200 |
|---|---|---|---|---|---|---|
| Light Clean | OFF | OFF | ON | ON | OFF | OFF |
| Medium Clean | ON | OFF | ON | ON | ON | OFF |
| Heavy Clean | ON | ON | ON | ON | ON | ON |
| Super Heat | ON | OFF | ON | ON | ON | ON |

Cleaning system 100 additionally may include a chemical disinfecting station 200. This station may be utilized in conjunction with physical disinfecting station 112 to kill all microorganisms on shopping cart 10 as well as work to prevent their growth and reproduction on shopping chart 10. Chemical disinfecting station 200 may include an enclosure 202, a first flex curtain 204 connected to an entrance of enclosure 202, a second flex curtain 206 connected to an exit of enclosure 202, and a disinfecting tank 208. Enclosure 202 may be large enough for one and no more than two shopping carts 10. Disinfecting tank 208 may contain liquid disinfectant and be connected to enclosure 202 and to control system 128.

Chemical disinfecting station 200 may include a control box 210. First, a customer may push shipping cart 10 past first flex curtain 204 and into enclosure 202. A red light on control box 210 may light when shopping cart 10 is positioned to receive liquid disinfectant. The customer may then push a start button 212. A timer 213 and a fan 214 inside enclosure 202 may start. A spray valve 216 may open to release chemical disinfectant. When timer 213 times out, a green light on control box 210 may light. The customer may then push shopping cart 10 out of enclosure 202 and past second flex curtain 206.

Disinfectant station 200 may be physically separated from frame 102. This may reduce an overall fixed size of cleaning system 100 and allow a non-sequential use of the work treating stations of cleaning system 100. For example, chemical disinfecting station 200 may be utilized before rinse station 108.

Chemical disinfecting station 200 may be a misting system that may apply a liquid disinfectant to a shopping cart 10. The liquid disinfectant may be any agent that may destroy or inactivate harmful microorganisms, such as such as bacteria, viruses, fungi, spores, and protozoa, and may include oxidants such as chlorine, chlorine dioxide, chloramines, and ozone, and may include hypochlorites, quaternary ammonium compounds, phenolic compounds, pine oil (at least 70%), alcohol, Dettol, hydrogen peroxide, iodine, potassium permanganate, Septustin M, toluene, or Virkon. A phenol coefficient is a measure of the bactericidal activity of a chemical compound in relation to phenol. Preferably, the liquid disinfectant may have a phenol coefficient greater than one.

The cleaning system 100 may operate as follows. As a shopping cart 10 may enter prewash 104 and a high presser wash may start and power wash shopping cart 10 cleaner. With shopping cart 10 moved to wash station 106 by conveyor 114, brushes 140 and 140 may start and wash shopping cart 10 clean. At rinse station 108, steam may start and steam clean shopping cart 10. After being dried in drying station 110, ultraviolet lights may kill the germs on shopping cart 10 at physical disinfecting station 112. A consumer may then push shopping cart 10 into chemical disinfecting station 200, where a mist may give a quick disinfectant spray to shopping cart 10.

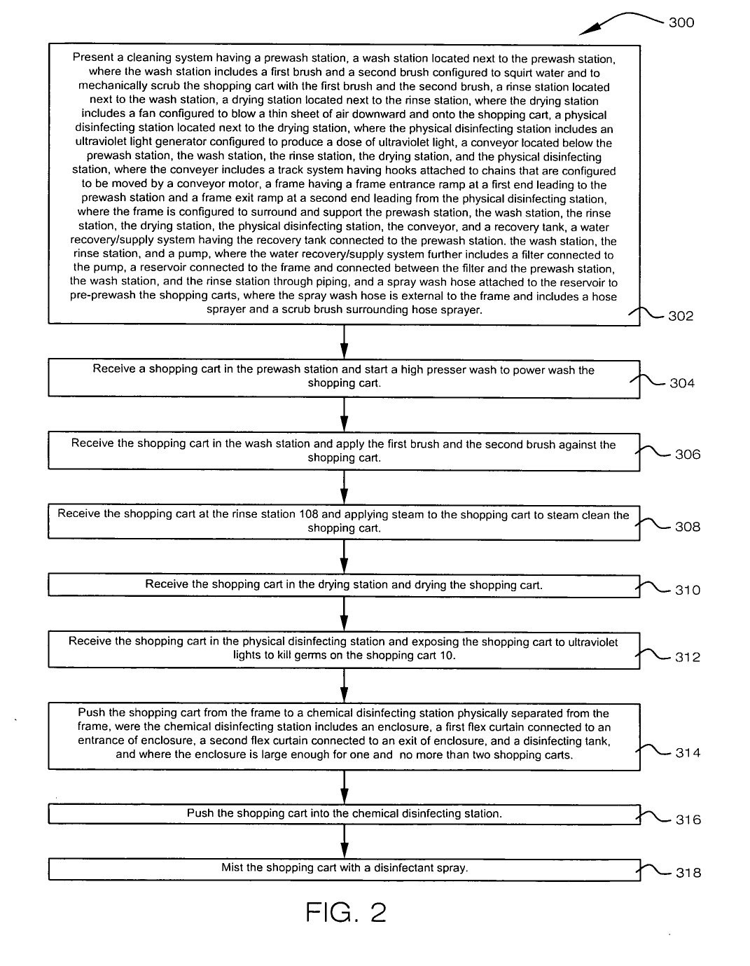

FIG. 2 is a method 300 to operate the cleaning system 100 for shopping carts 10. At step 302, method 300 may present a cleaning system having a prewash station, a wash station located next to the prewash station, where the wash station includes a first brush and a second brush configured to squirt water and to mechanically scrub the shopping cart with the first brush and the second brush, a rinse station located next to the wash station, a drying station located next to the rinse station, where the drying station includes a fan configured to blow a thin sheet of air downward and onto the shopping cart, a physical disinfecting station located next to the drying station, where the physical disinfecting station includes an ultraviolet light generator configured to produce a dose of ultraviolet light, a conveyor located below the prewash station, the wash station, the rinse station, the drying station, and the physical disinfecting station, where the conveyer includes a track system having hooks attached to chains that are configured to be moved by a conveyor motor, a frame having a frame entrance ramp at a first end leading to the prewash station and a frame exit ramp at a second end leading from the physical disinfecting station, where the frame is configured to surround and support the prewash station, the wash station, the rinse station, the drying station, the physical disinfecting station, the conveyor, and a recovery tank, a water recovery/supply system having the recovery tank connected to the prewash station, the wash station, the rinse station, and a pump, where the water recovery/supply system further includes a filter connected to the pump, a reservoir connected to the frame and connected between the filter and the prewash station, the wash station, and the rinse station through piping, and a spray wash hose attached to the reservoir to pre-prewash the shopping carts, where the spray wash hose is external to the frame and includes a hose sprayer and a scrub brush surrounding hose sprayer.

At step 304, method 300 may receive a shopping cart in the prewash station and start a high presser wash to power wash the shopping cart. At step 306, method 300 may receive the shopping cart in the wash station and apply the first brush and the second brush against the shopping cart. At step 308, method 300 may receive the shopping cart at the rinse station 108 and applying steam to the shopping cart to steam clean the shopping cart.

At step 310, method 300 may receive the shopping cart in the drying station and drying the shopping cart. At step 312, method 300 may receive the shopping cart in the physical disinfecting station and exposing the shopping cart to ultraviolet lights to kill germs on the shopping cart 10.

At step 314, method 300 may push the shopping cart from the frame to a chemical disinfecting station physically separated from the frame, were the chemical disinfecting station includes an enclosure, a first flex curtain connected to an entrance of enclosure, a second flex curtain connected to an exit of enclosure, and a disinfecting tank, and where the enclosure is large enough for one and no more than two shopping carts. At step 316, method 300 may push the shopping cart into the chemical disinfecting station and at step 318, method 300 may mist the shopping cart with a disinfectant spray.

The cleaning system may be a cleaning system for shopping carts. The system may include of an elongated structure made of stainless steel. The lower portion of the cleaning system may feature a track system, the center of which may be equipped with a chain-based conveyor system that may pull carts through the unit.

The first station may consist of a series of high-pressure water jets that may apply a water/soap solution. The next station may feature brushes of some type that may mechanically scrub all or a portion of a cart. After this may be a high pressure, hot water rinse system. The first and second stations may be supplied via high pressure pumps located at some point in or on the unit's housing. The lower portion of the unit may presumably feature drain fittings. The unit may feature a water recovery system, similar to those employed in many car washes. The next station may consist of an air knife-based drying system. This may be followed by an ultraviolet light-based disinfecting station. The last station may consist of a misting system that may apply a liquid disinfectant.

The cleaning system may fulfill a need for an improved method of cleaning shopping carts. Appealing features of the cleaning system may include its efficiency, ease of operation, adaptability, and the improved level of hygiene it may provide. This system may be adapted for use with most typical shopping carts and may be set up at a variety of possible locations around a retail establishment. In operation, the cleaning system may provide a fast and thorough method of dislodging dirt, grease, bacteria, and other contaminants from shopping carts, thereby improving their appearance and helping prevent the spread of disease.

The cleaning system may be an automatic or manual sanitizer or disinfected cart cleaner incorporating a presser wash, steam, and ultraviolet light. The cleaning system may include an entrance and exit such that upon the entrance of the cart mechanical or manual the wash disinfectant or sanitizer may take place as the cart enters the washer it may start and end after the carts exit with a clean sanitized or disinfected cart.

The cleaning system generally may relate to devices and techniques to protect one from the handling of carts resulting in the exposure to germs, viruses, and infectious diseases. Having incorporated therein a sanitizer, disinfector cart wash. Contaminated carts carry a host of infectious agents on the surface of the cart. Handling carts with these infectious agents on the surface occur to more then a million shoppers every day, and for many young or old it is a death sentence.

The shopping carts may be manually or mechanically pushed or pull through the housing of the cleaning system. The cleaning system may utilize several different applications to render the shopping cart sanitized. The cleaning system may have at least three of the following controls, switches, valves and solenoids: timer, selector switch, water inlet valve, floor switch, soap dispenser, dissecting dispenser, drain solenoid, thermostat, door switch, drying fan, heating element, upper and/or lower spray arms. The cleaning system may utilize one or have all inaugurated into one housing. As the shopping cart enter the wash, 1. High presser wash may start and powers wash the shopping cart cleaner. 2. Brushes may start and wash the shopping cart clean. 3. steam may star and steam clean the carts. 4. Ultraviolet lights may kin the germs on the cart. 5. A mist box that gives a quick disinfectant spray.

The cleaning system may assist in helping to alleviate the problem in the general public of spreading infectious diseases derived from inadvertent exposure of the general public resulting from the use of the common shopping cart. The cleaning system may overcome the presently known problems related to the inadvertent transfer of viruses and diseases to the general public, resulting from the use of a contaminated shopping cart.

The information disclosed herein is provided merely to illustrate principles and should not be construed as limiting the scope of the subject matter of the terms of the claims. The written specification and figures are, accordingly, to be regarded in an illustrative rather than a restrictive sense. Moreover, the principles disclosed may be applied to achieve the advantages described herein and to achieve other advantages or to satisfy other objectives, as well.

What is claimed is:

1. A cleaning system for shopping carts, the cleaning system comprising:
a prewash station;
a wash station located next to the prewash station, where the wash station includes a first brush and a second brush configured to squirt water and to mechanically scrub the shopping cart with the first brush and the second brush;
a rinse station located next to the wash station;
a drying station located next to the rinse station, where the drying station includes a fan configured to blow a thin sheet of air downward and onto the shopping cart;
a physical disinfecting station located next to the drying station, where the physical disinfecting station includes an ultraviolet light generator configured to produce a dose of ultraviolet light;
a conveyor located below the prewash station, the wash station, the rinse station, the drying station, and the physical disinfecting station, where the conveyer includes a track system having hooks attached to chains that are configured to be moved by a conveyor motor;
a frame having a frame entrance ramp at a first end leading to the prewash station and a frame exit ramp at a second end leading from the physical disinfecting station, where the frame is configured to surround and support the prewash station, the wash station, the rinse station, the drying station, the physical disinfecting station, the conveyor, and a recovery tank;
a water recovery/supply system having the recovery tank connected to the prewash station, the wash station, the rinse station, and a pump, where the water recovery/supply system further includes a filter connected to the pump, a reservoir connected to the frame and connected between the filter and the prewash station, the wash station, and the rinse station through piping; and
a spray wash hose attached to the reservoir to pre-prewash the shopping carts, where the spray wash hose is external to the frame and includes a hose sprayer and a scrub brush surrounding hose sprayer.

2. The cleaning system of claim 1, where the prewash station includes high pressure water jets configured to contact a shopping cart with a water/soap solution to loosen undesired matter from the shopping cart.

3. The cleaning system of claim 1, further comprising:
a cleaning fluid tank attached external to and between the prewash station and the wash station, where the cleaning fluid tank is partitioned into two compartments and each compartment includes a different cleaning fluid.

4. The cleaning system of claim 1, where the first brush of the wash station is attached to water station head through one link and the second brush is attached to the water station head through four links, where each of the four links is configured to move relative to the other links between the second brush and the water station head, were the first brush and the second brush are configured to rotate.

5. The cleaning system of claim 4, further comprising:
a control system, where the control system is configured to independently control each link in the wash station to move the first brush and the second brush along predetermined paths; and
a plurality of light sensors, where the prewash station, the wash station, the rinse station, the drying station, and the physical disinfecting station each include a light sensor, where each light sensor is configured to identify whether a shopping cart is located within the station associated with the light sensor and provide information to the control system to the permit control system to identify a horizontal position of the shopping cart relative to the prewash station, the wash station, the rinse station, the drying station, and the physical disinfecting station.

6. The cleaning system of claim 5, further comprising:
a plurality of station valves, where each of the prewash station, the wash station, and the rinse station are attached to a station valve and where each station valve is in communication with the control system.

7. The cleaning system of claim 1, where the reservoir is configured to receive clean water from the filter and through a reservoir valve attached to the reservoir.

8. The cleaning system of claim 1, where the reservoir is configured to be under fluid pressure from the pump where this fluid pressure is configured to cause the first brush and the second brush of the wash station to rotate.

9. The cleaning system of claim 1, where the rinse station includes a rinse station head having a first spray nozzle and a second spray nozzle, where the rinse station head is configured to rotate as rinsing water exits the first spray nozzle and the second spray nozzle.

10. The cleaning system of claim 1, where the dose of ultraviolet light of the physical disinfecting station as applied against each surface of the shopping cart is not less than 16.2 mJ/cm² and may greater than 20.0 mJ/cm².

11. The cleaning system of claim 1, where the chains of the conveyor are endless and the conveyor motor is a variable speed motor.

12. The cleaning system of claim 1, further comprising:
a control switch box configured to be in communication with the control system, where the control switch box includes a control knob to dial in various mode of operations, a key pad to enter access codes and number of carts, a status indicator light, and a number of carts display, where the control switch box is mounted on the frame at a location adjacent to the first end.

13. The cleaning system of claim 12, where the mode of operations include a light clean, medium clean, heavy clean, and a super heat mode of operations, and where each mode of operation is configured to be set through the control switch box relative to a station according to the following table:

| | Station: | | | | |
|---|---|---|---|---|---|
| | | | | | Physical |
| | Prewash | Wash | Rinse | Drying | disinfecting |
| Light Clean mode | OFF | OFF | ON | ON | OFF |
| Medium Clean mode | ON | OFF | ON | ON | ON |
| Heavy Clean mode | ON | ON | ON | ON | ON |
| Super Heat mode | ON | OFF | ON | ON | ON |

14. The cleaning system of claim 1, further comprising:
a chemical disinfecting station having an enclosure, a first flex curtain connected to an entrance of enclosure, a second flex curtain connected to an exit of enclosure, and a disinfecting tank, where the enclosure is large enough for one and no more than two shopping carts, where the chemical disinfecting station is physically separated from the frame.

15. A method to operate a cleaning system for shopping carts, the method comprising:
presenting a cleaning system having a prewash station, a wash station located next to the prewash station, where the wash station includes a first brush and a second brush configured to squirt water and to mechanically scrub the shopping cart with the first brush and the second brush, a rinse station located next to the wash station, a drying station located next to the rinse station, where the drying station includes a fan configured to blow a thin sheet of air downward and onto the shopping cart, a physical disinfecting station located next to the drying station, where the physical disinfecting station includes an ultraviolet light generator configured to produce a dose of ultraviolet light, a conveyor located below the prewash station, the wash station, the rinse station, the drying station, and the physical disinfecting station, where the conveyer includes a track system having hooks attached to chains that are configured to be moved by a conveyor motor, a frame having a frame entrance ramp at a first end leading to the prewash station and a frame exit ramp at a second end leading from the physical disinfecting station, where the frame is configured to surround and support the prewash station, the wash station, the rinse station, the drying station, the physical disinfecting station, the conveyor, and a recovery tank, a water recovery/supply system having the recovery tank connected to the prewash station, the wash station, the rinse station, and a pump, where the water recovery/supply system further includes a filter connected to the pump, a reservoir connected to the frame and connected between the filter and the prewash station, the wash station, and the rinse station through piping, and a spray wash hose attached to the reservoir to pre-prewash the shopping carts, where the spray wash hose is external to the frame and includes a hose sprayer and a scrub brush surrounding hose sprayer;
receiving a shopping cart in the prewash station and starting a high presser wash to power wash the shopping cart;
receiving the shopping cart in the wash station and applying the first brush and the second brush against the shopping cart;
receiving the shopping cart at the rinse station and applying steam to the shopping cart to steam clean the shopping cart;
receiving the shopping cart in the drying station and drying the shopping cart; and
receiving the shopping cart in the physical disinfecting station and exposing the shopping cart to ultraviolet lights to kill germs on the shopping cart.

16. The method of claim 15, further comprising:
pushing the shopping cart from the frame to a chemical disinfecting station physically separated from the frame, were the chemical disinfecting station includes an enclosure, a first flex curtain connected to an entrance of enclosure, a second flex curtain connected to an exit of enclosure, and a disinfecting tank, and where the enclosure is large enough for one and no more than two shopping carts; and
pushing the shopping cart into the chemical disinfecting station; and
misting the shopping cart with a disinfectant spray.
